(19) United States
(12) Patent Application Publication
(10) Pub. No.: US 2022/0370115 A1
(43) Pub. Date: Nov. 24, 2022

(54) ELECTROSURGICAL GENERATOR

(71) Applicant: OLYMPUS WINTER & IBE GMBH, Hamburg (DE)

(72) Inventors: Stefan DIETRICH, Berlin (DE); Jelle DIJKSTRA, Berlin (DE); Thomas FAEHSING, Berlin (DE)

(73) Assignee: OLYMPUS WINTER & IBE GMBH, Hamburg (DE)

(21) Appl. No.: 17/771,607

(22) PCT Filed: Oct. 23, 2020
(86) PCT No.: PCT/EP2020/079951

§ 371 Date: Apr. 25, 2022

(30) Foreign Application Priority Data
Oct. 24, 2019 (DE) 10 2019 128 792.8

(51) Int. Cl. A61B 18/12

(52) U.S. Cl. CPC A61B 18/1206; A61B 2017/00137

(57) ABSTRACT

An electrosurgical generator comprising two HF generator units, two output transformers, and at least four output terminals for connecting electrosurgical instruments. Together with the associated output transformer and corresponding output terminals, each HF generator unit forms an HF generator module. The electrosurgical generator comprises a control unit connected to two HF generator modules and configured to provide the following operating modes: parallel mode in which two output transformers are connected in parallel so that an electrosurgical instrument can be operated with higher output current, series mode in which two output transformers are connected in series so that, via the output terminals, an electrosurgical instrument can be operated with higher output voltage, and multi-instrument mode in which output transformers are each connected independently to pair of output terminals for respectively one electrosurgical instrument in which output terminal of generator module is electrically connected to output terminal of respective other generator module.

The invention relates to an electrosurgical generator comprising output terminals for connecting at least one electrosurgical instrument, wherein the output terminals are connected to a secondary winding of an output transformer.

By means of electrosurgery, biological tissue—i.e. body tissue—can be cut, coagulated (cauterized) and/or vaporized. Electrosurgery typically uses high-frequency alternating currents with a frequency between 0.2 MHz and 3 MHz.

An electrosurgery system typically includes an electrosurgical generator for generating the high-frequency alternating current. The electrosurgical generator typically has two output terminals to which an electrosurgical instrument can be connected and between which a high frequency AC voltage is provided during operation. The output terminals are electrically connected to an output transformer on its secondary side. For generating a high-frequency alternating voltage or high-frequency alternating current for operating an electrosurgical instrument connected to the output terminals, the electrosurgical generator has an HF generator unit which is galvanically connected to a primary winding of the output transformer, so that a resonant circuit is formed whose component is the primary side of the output transformer. The output transformer galvanically isolates the output terminals and an electrosurgical instrument connected to them during operation from the HF generator unit.

Such an electrosurgical generator serves the purpose of supplying a electrosurgical instrument connected to the output terminals with a high-frequency alternating current so that the electrosurgical instrument can be used for a desired electrosurgical application such as cutting or ablating body tissue. The various electrosurgical applications typically require high-frequency alternating currents, which differ in terms of power requirements or voltage depending on the application. This is why known electrosurgical generators typically provide different operating modes. There are, for example, operating modes in which the electrosurgical generator requires a very high peak voltage between 4 KV and 5 KV, as is necessary for certain coagulations. Another operating mode requires very high currents, for example for cutting tissue when a saline solution is used. An example of an electrosurgical generator having three output transformers for three different operation modes is disclosed in DE 29 01 153.

In order to provide such different operating modes, known electrosurgical generators have an output transformer where windings can be switched on the primary and/or secondary side. Other known electrosurgical generators have several independent transformers, for example one transformer for generating a high output voltage and another one for supplying a high output current.

The invention is based on the object of creating an electrosurgical generator that provides different operating modes and can be realized with limited material expenditure.

According to the invention, this object is achieved by an electrosurgical generator that comprises at least two separate HF generator units and at least two separate output transformers as well as a total of at least four output terminals for connecting at least two electrosurgical instruments. Two output terminals are respectively connected to the secondary winding of a corresponding output transformer in such a way that one of the output terminals is used for connecting a working electrode and the other of the two output terminals is used for connecting a neutral electrode. Together with the associated output transformer and the corresponding output terminals, each of the HF generator units respectively forms an HF generator module.

The electrosurgical generator according to the invention comprises a control unit that is connected to the at least two HF generator modules and configured to provide the following operating modes:
a parallel mode in which the at least two output transformers are connected in parallel at their secondary sides so that, via the output terminals, an electrosurgical instrument can be operated with a higher output current than the current that would be able to be provided by one HF generator module alone,
a serial mode in which the at least two output transformers are connected in series at their secondary sides so that, via the output terminals, an electrosurgical instrument can be operated with a higher output voltage than the voltage that would be able to be provided by one HF generator module alone, and
a multi-instrument mode in which the output transformers are each connected independently of each other to a pair of output terminals for respectively one electrosurgical instrument. In a special case, the two neutral electrodes can be connected to one another.

Optionally, the control unit may be configured to provide a multi-electrode mode, in which an output terminal of a generator module is electrically connected to an output terminal of another generator module, which results in a total of three output terminals that are each connected to an electrode of an electrosurgical instrument.

If such an electrosurgical instrument for instance comprises two generator modules, their use according to the invention thus allows simultaneous activation of two outputs with medium demand (current, voltage, power, e.g. 120 W) and, alternatively, an activation of one output with high current or high voltage and/or high power.

To this end, such an electrosurgical generator has at least two independent HF generator modules that do not need to be oversized and do not require any additional components, wherein the HF generator modules—respectively formed by one HF generator unit and the associated output transformer—may, by means of the control unit, be connected in parallel or in series as needed. Thus, for instance instead of building two HF generator modules, each of which would be able to supply both the required maximum current and the required maximum voltage, the invention provides for at least two independent HF generator modules that can supply a desired maximum current or a desired maximum voltage through a corresponding connection of the outputs. In a standard application (single output), two HF generator modules can be connected either in series or in parallel, in order to be able to supply the required voltage or the required current. In a dual instrument mode, the HF generator modules can be operated independently of each other, but only with half the maximum current and/or half the maximum voltage, which is sufficient for most applications where two electrosurgical instruments are to be used in parallel.

Since the HF generator modules are controlled by one shared control unit, the high-frequency alternating currents on the secondary side of the output transformers can be matched to each other in particular also in a multi instrument mode, in order to avoid for example cross currents. In addition, such an electrosurgical generator makes it also possible to operate electrosurgical instruments with three electrodes.

Preferably, the electrosurgical generator has at least three switches that are connected to the output terminals of at least two HF generator modules in such a way that two output terminals of different HF generator modules for a working electrode and two output terminals of the different HF generator modules for a neutral electrode can respectively be electrically connected to each other for the parallel mode through the closing of two of the three switches, so that the two HF generator modules are connected in parallel at their outputs. For the series mode, an output terminal for a working electrode of a generator module and an output terminal for a neutral electrode of another generator module can be electrically connected to each other through the closing of the third switch, so that the two HF generator modules and their outputs are connected in series. For a dual instrument mode, all three switches are open and the two HF generator modules can be operated independently of each other. However, since in this mode as well, the at least two HF generator modules are preferably controlled by the one control unit, the operation of the two HF generator units, that are generally independent of each other, can be coordinated via the control unit in such a way that cross currents, for example, are avoided and that an instrument with more than two electrodes can be reasonably operated.

The control of the switches and the corresponding switching of the switches is preferably carried out by the control unit, which is connected to the at least three switches for this purpose and is thus able to open and close the switches in accordance with the operating mode selected in the respective case.

The switches may be mechanical switches such as relays, but may also be formed by semiconductors, for example by field effect transistors or the like.

As already suggested above, for controlling the HF generator units, the control unit is preferably connected to each of the at least two HF generator units. This allows the control unit to also influence the respective generation of a high-frequency alternating current at the output of a respective output transformer, for example in order to coordinate the generated high-frequency alternating currents in the phase and to thus prevent cross currents.

Preferably, the control unit is furthermore configured to provide two versions of the multi-instrument mode, namely a first version in which the output terminals for connecting a neutral electrode are not electrically connected to each other, and a second version in which the output terminals for connecting a neutral electrode are electrically connected to each other.

In one embodiment of the electrosurgical generator, the at least two output terminals for connecting a neutral electrode are electrically connected to each other and can, for example, form a joint neutral terminal.

The output transformers are each preferably connected on the primary side to one of the HF generator units in such a way that, together with the associated output transformer, the respective HF generator unit forms a corresponding resonant circuit, wherein a primary winding of the respective output transformer is part of the corresponding resonant circuit.

In another advantageous embodiment, the control unit is configured to provide a “multi-electrode mode” in which an output terminal of a generator module is electrically connected to an output terminal of another generator module, which results in a total of three output terminals that are each connected to an electrode of an electrosurgical instrument. In the “multi-electrode mode”, it is possible to either operate one single electrosurgical instrument with three electrodes or three individual probes, such as those used for example for tumor ablation, in order to coagulate a large volume. In the “multi-electrode mode”, the two output terminals for connecting a neutral electrode are preferably electrically connected to each other.

The control unit is preferably configured to operate the at least two HF generator modules in the “multi-electrode mode” with different phase positions.

The HF generator modules are preferably configured such that the maximum output voltage per generator module is 2,500 Vₚₑₐₖ—and thus about half of the maximum output voltage of 4,200 Vₚₑₐₖ.

Furthermore, the HF generator modules are preferably configured such that the maximum output current is 4.5 Aᵣₘₛ.

Finally, the HF generator modules are preferably configured such that their maximum frequency range comprises 40 kHz to 1 MHz and thus the spectrum from ultrasound to HF.

The control unit is preferably configured to control the phase position between the currents and/or voltages output by the two HF generator modules.

In the following, the invention shall be explained in more detail based on an exemplary embodiment with reference to the figures.

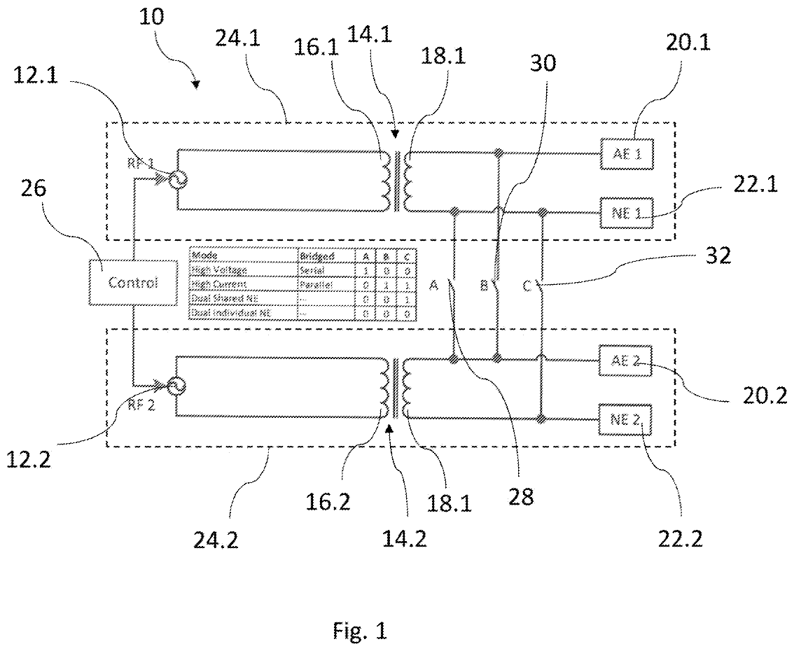

FIG. 1: shows an electrosurgical generator according to the invention having two generator modules;

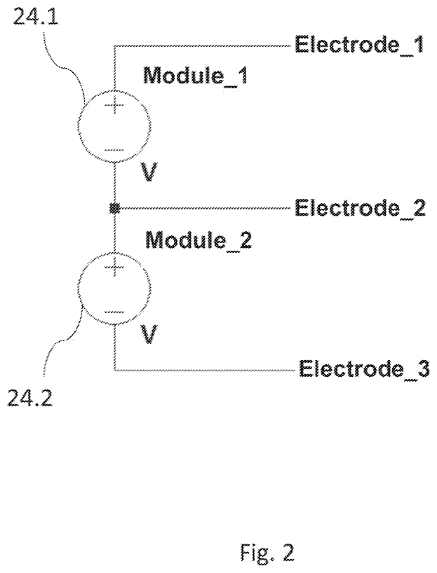

FIG. 2: illustrates the operation of the electrosurgical generator of FIG. 1 in a “multi-electrode mode”; and

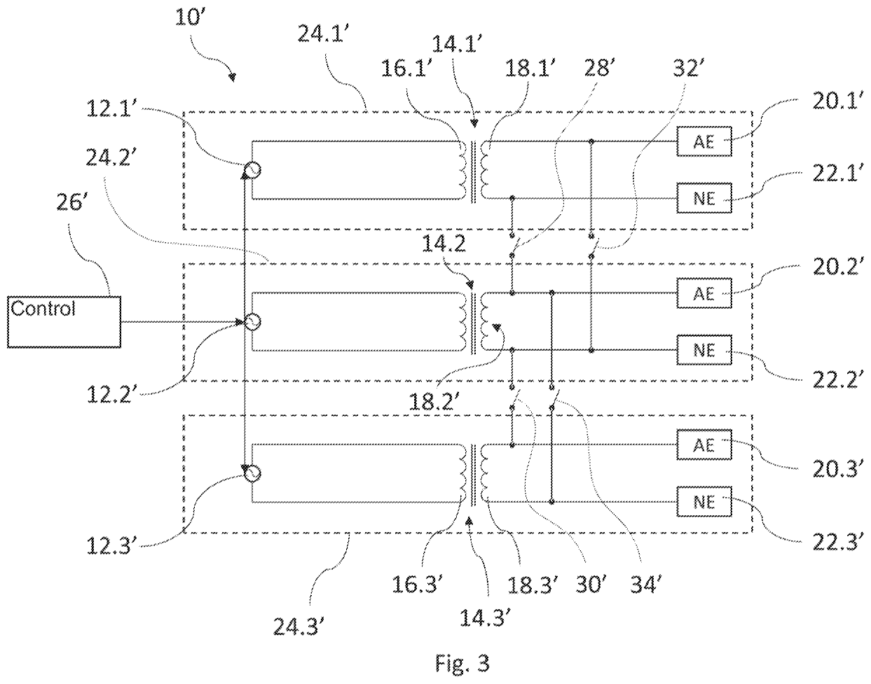

FIG. 3: an electrosurgical generator according to the invention with three generator modules;

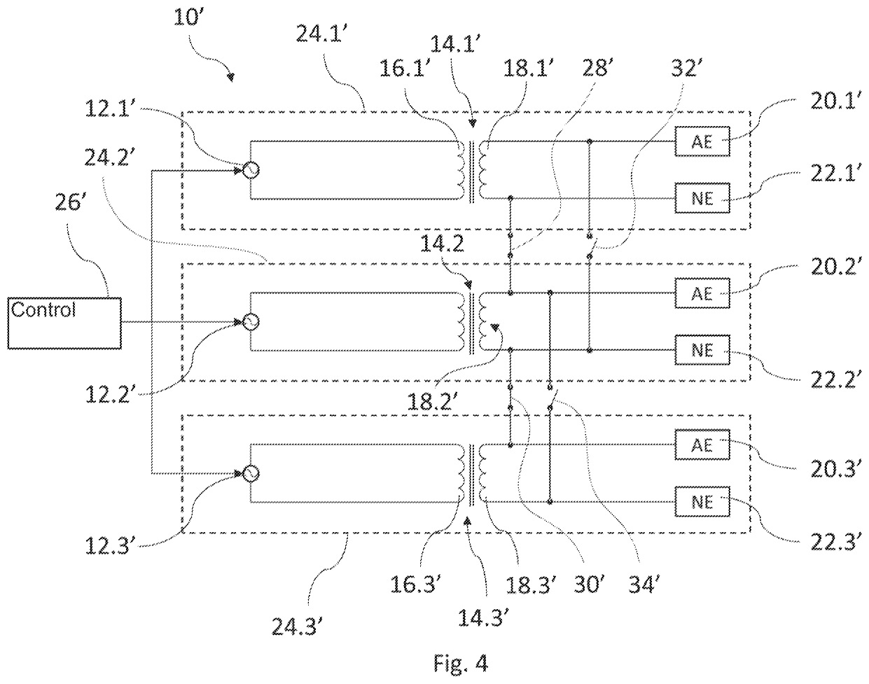

FIG. 4: the electrosurgical generator of FIG. 3 with secondary windings of all three generator modules connected in series;

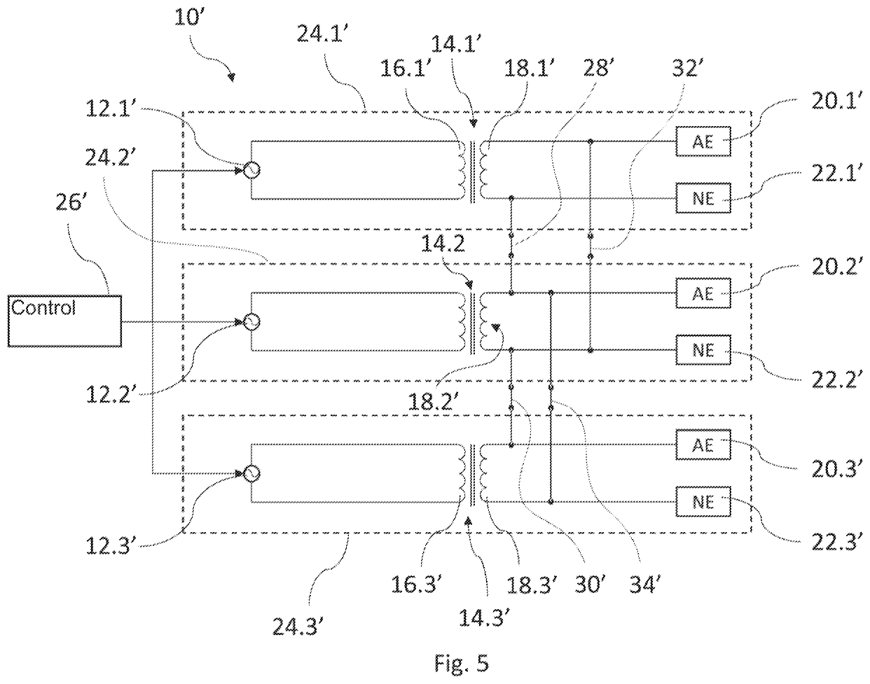

FIG. 5: the electrosurgical generator of FIG. 3 with all three generator modules connected in parallel;

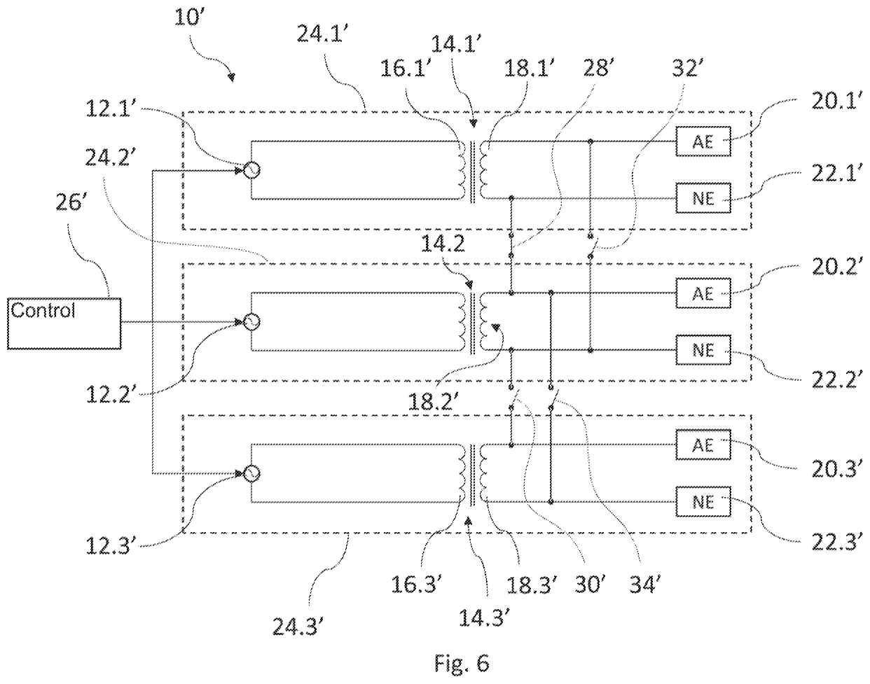

FIG. 6: the electrosurgical generator of FIG. 3 with secondary windings of two of the three generator modules connected in series;

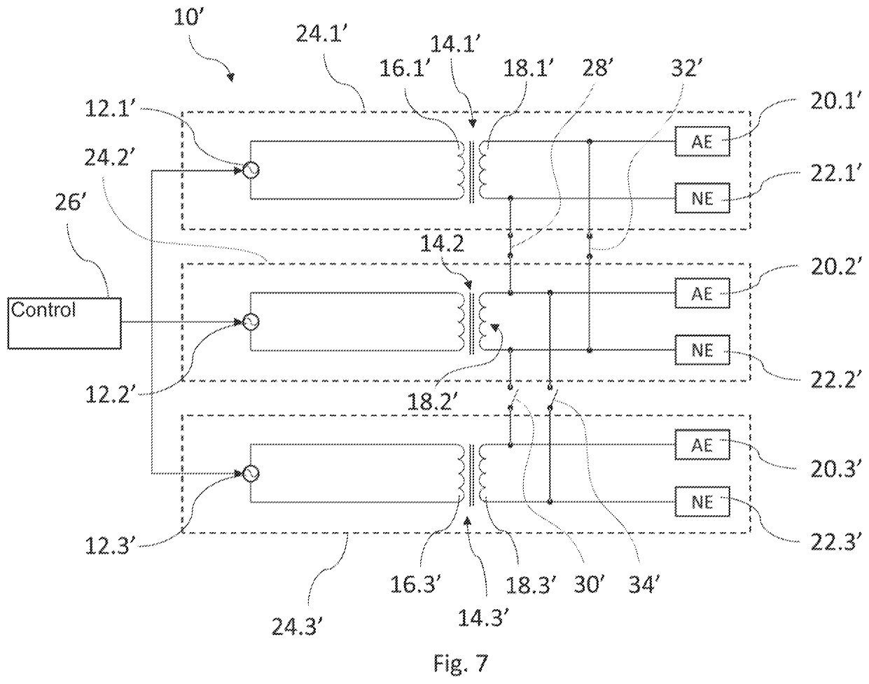

FIG. 7: the electrosurgical generator of FIG. 3 with secondary windings of two of the three generator modules connected in parallel; and

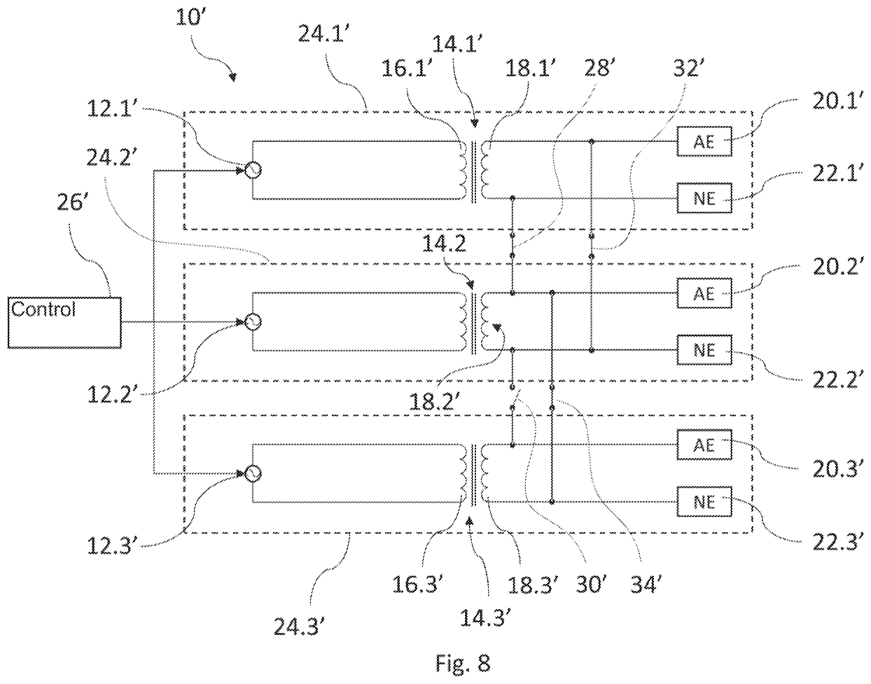

FIG. 8: the electrosurgical generator of FIG. 3 with secondary windings of two of the three generator modules connected in parallel and a third generator module connected in series.

FIG. 1 shows an electrosurgical generator 10, comprising two HF generator units 12.1 and 12.2, and two output transformers 14.1 and 14.2.

Each of the two output transformers 14.1 and 14.2 comprises a primary winding 16.1 or 16.2, respectively, on the primary side and a secondary winding 18.1 or 18.2, respectively, on the secondary side. The respective HF generator unit 12.1 or 12.2 is connected to the corresponding primary winding 16.1 or 16.2, respectively, of the associated output transformer 14.1 or 14.2, respectively, so that, in each case, one HF generator unit 12.1 or 12.2 forms a resonant circuit with the associated primary winding 16.1 or 16.2, respectively, of the associated output transformer 14.1 or 14.2, respectively.

In each case, two output terminals 20.1 and 22.1 or 20.2 and 22.2, respectively, are connected to the respective secondary winding 18.1 or 18.2 of the output transformer 14.1 or 14.2, respectively. One of these output terminals forms in each case an output terminal 20.1 or 20.2, respectively, for connecting a working electrode of an electrosurgical instrument, while the respective other output terminal 22.1 or 22.2, respectively, forms an output terminal for connecting a neutral electrode of the respective electrosurgical instrument.

With the output transformer 14.1 or 14.2, respectively, connected to it, and the associated output terminals 20.1 and 22.1 or 20.2 and 22.2, respectively, the respective HF generator unit 12.1 or 12.2 forms a generator module 24.1 or 24.2, respectively.

Both HF generator modules 24.1 and 24.2 are basically independent of one another, but are controlled by a shared control unit 26. To this end, the control unit 26 is effectively connected both to the HF generator unit 12.1 and the generator unit 12.2, and is thus able to control the corresponding HF generator module 24.1 and 24.2.

Via a total of three switches 28, 30 and 32, the output terminals 20.1, 22.1, 20.2 and 22.2 of the HF generator modules 24.1 and 24.2 may be connected either parallel to each other or alternatively in series. To this end, the switch 28 is, on the one hand, electrically connected to the first generator module's 24.1 output terminal 22.1 for connecting a neutral electrode, and, on the other, electrically connected to the second HF generator module's 24.2 output terminal 20.2 for connecting a working electrode. Through the closing of the switch 28, the outputs of the two generator modules 24.1 and 24.2 can be connected in series so that an instrument can, for example, be connected to the output terminal 20.1 of the first generator module 24.1 and the output terminals 22.2 of the HF generator module 24.2, and can thus be operated at a voltage that is twice as high as the maximum output voltage of one of the HF generator modules 24.1 or 24.2. The switch 28 is effectively connected to the control unit 26 so that the switch 28 can be opened and closed.

The closing of the switch 28, alone, makes it possible to also operate electrosurgical instruments with three electrodes by means of such an electrosurgical generator. Thus, in addition to the operating modes “parallel mode”, “serial mode” and “multi-instrument mode”, a “multi-electrode mode” can be realized as well. Said multi-electrode mode can be used both with a single instrument with three electrodes or with three individual probes, such as those that are used in tumor ablation, in order to coagulate a large volume.

If, through the closing of the switch 28, the electrodes are connected as shown in FIG. 2, any voltage can be set between each pair of electrodes. If, for example, the amplitudes and phases of the two HF generator modules 24.1 and 24.2 are the same, single voltage will respectively be applied between the electrodes 1 and 2 and/or 2 and 3, and double voltage between electrodes 1 and 3. If the amplitudes are the same, but the phase is turned by 180°, single voltage will respectively be applied between electrodes 1 and 2 and/or 2 and 3, and no voltage will be applied between electrodes 1 and 3.

With the help of the two switches 30 and 32, the two HF generator modules 24.1 and 24.2 can be connected in parallel to each other, so that an electrosurgical instrument can be operated with a current that is twice as high as the current that one of the two HF generator modules 24.1 or 24.2 can supply on its own. To this end, the switch 30 is electrically connected to the two output terminals 20.1 and 20.2 for connecting the working electrode of an electrosurgical instrument, while the switch 32 is electrically connected to the two output terminals 22.1 and 22.2 for connecting the respective neutral electrode of an electrosurgical instrument. When both switches 30 and 32 are closed and the switch 28 is open, the two HF generator modules 24.1 and 24.2 are connected in parallel via their outputs. In order to be able to achieve this, the two switches 30 and 32, as well, are connected to the control unit 26.

When all three switches 28, 30 and 32 are open, the two HF generator modules 24.1 and 24.2 can be operated independently of each other. However, since the respective HF generator units 12.1 and 12.2 are each connected to the control unit 26, the control unit 26 is able to control the two HF generator modules 24.1 and 24.2 also in the module instrument mode, in which all three switches 28, 30 and 32 are open, in such a way that the voltages are, for example with regard to the phase, coordinated via the output terminals 20.1 and 22.1 or 20.2 and 22.2, respectively, in such a way that no cross currents occur during operation with two instruments. Finally, by only closing the switch 32, the control unit 26 can cause the neutral electrodes of two electrosurgical instruments connected to the electrosurgical generator 10 to have the same potential.

Thus, by switching the switches 28, 30 and 32 accordingly, the control unit 26 can switch the electrosurgical generator 10 in a total of four different operating modes. A first operating mode is a series mode, in which the two HF generator modules 24.1 and 24.2 are connected to each other in series. This is the case when the control unit 26 closes the switch 28 and opens the two switches 30 and 32. Another operating mode is a parallel mode, in which the two HF generator modules 24.1 and 24.2 are connected to each other in parallel. In order to establish this parallel mode, the switch 28 is opened by the control unit 26, while the two switches 30 and 32 are closed by the control unit 26. If the control unit 26 opens all three switches 28, 30 and 32, a dual-instrument mode is established, in which the two HF generator modules 24.1 and 24.2 are, on their output side, electrically independent of each other. If the control unit 26 only closes the switch 32, a dual-instrument mode is established, in which the output terminals 22.1 and 22.2 for connecting a respective neutral electrode of an electrosurgical instrument are at the same potential.

The two HF generator modules 24.1 and 24.2 are configured such that the maximum output voltage per generator module is 2,500 Vₚₑₐₖ and thus about half of the maximum output voltage of 4,200 Vₚₑₐₖ. In addition, the two HF generator modules 24.1 and 24.2 are configured such that their maximum output current is 4.5 Aᵣₘₛ and their maximum frequency range comprises 40 kHz to 1 MHz and thus the spectrum from ultrasound to HF.

The control unit 26 is configured to control the amplitude and the phase position between the currents and/or voltages output by the two HF generator modules 24.1 and 24.2.

This means that the phase position is determined by the control unit 26 and variable. If, for example, a three-electrode instrument (NE1/AE/NE2—3 electrical contacts) is connected to the two generator modules 24.1 and 24.2, changing the phase position makes it possible to switch between the cutting mode for cutting tissue (Cut) and a coagulation mode for coagulating tissue (Coag). For the coagulation mode, the phase angle (phase position) is 0°, for the cutting mode, it is 180°. In addition, future instruments can benefit from a change of the phase position.

Thus, the electrosurgical generator 10 offers a plurality of operating modes, and can, depending on what is needed, either provide a high output voltage or a high output current, without any of its HF generator modules 24.1 or 24.2 having to be configured for the maximum output voltage or the maximum output current to be provided by the electrosurgical generator 10.

An electrosurgical generator 10′ can also have more than two generator modules, for example three generator modules 24.1, 24.2′ and 24.3′; see FIG. 3.

In the electrosurgical generator 10′, the output transformers 14.1′, 14.2′ and 14.3′ can also be connected in series on the secondary side, connected in parallel or operated separately. For this purpose, four switches are provided in the example, namely switches 28′, 30′, 32′ and 34′.

In FIG. 3, all switches 28′, 30′, 32′ and 34′ are open, so that the three generator modules 24.1′, 24.2′ and 24.3′ are independent of each other on their output sides. However, the respective associated HF generator units 12.1′, 12.2′ and 12.3′ are controlled by the control unit 26′ so that, similar to the embodiment example according to FIG. 1, a phase angle can be set between the high-frequency alternating voltages generated by the HF generator units 12.1, 12.2 and 12.3.

Incidentally, the control unit 26 is also operatively connected to the switch 28′, 30′, 32′ and 34′ so that these switches can be controlled, closed or opened by the control unit 26.

In the example shown in FIG. 3, as already mentioned, all of the four switches 28′, 30′, 32′ and 34′ are open.

When switches 28′ and 30′ are closed but switches 32′ and 34′ are open, the three output transformers 14.1′, 14.2′ and 14.3′ of generator modules 24.1′, 24.2′ and 24.3′ are connected in series with each other on their secondary side. This means that the secondary windings 18.1′, 18.2′ and 18.3′ are connected in series with each other when the switches 28′ and 30′ are closed but the switches 32′ and 34′ are open. This is shown as an example in FIG. 4. By connecting the secondary windings 18.1′, 18.2′ and 18.3′ in series, an increased output voltage is present between the output terminals 20.1 and 22.3, which is approximately equal to the sum of the output voltages of the individual output transformers 14.1′, 14.2′ and 14.3′, for example. To take advantage of this increased output voltage, an electrosurgical instrument can be connected to the output terminals 20.1′ and 22.3′.

To achieve an increased output current, the three generator modules 24.1′, 24.2′ and 24.3′ can also be connected in parallel on the output side. Such a parallel connection exists when the switches 28′, 30′, 32′ and 34′, controlled by the control unit 26′, are all closed. This is shown by way of example in FIG. 5. When the switches 28′, 30′, 32′ and 34′ are closed, the secondary windings 18.1′, 18.2′ and 18.3′ of the output transformers 14.1′, 14.2′ and 14.3′ are connected in parallel with each other. In the illustrated embodiment, the HF generator unit 12.2′ is operated with a phase position shifted by 180° to produce the desired effect of increased output current, because the arrangement of the switches 28′, 30′, 32′ and 34′ causes the phase position of the output transformer 14.2′ to be rotated by 180° when the switches are closed in relation to the phase position of the output transformers 14.1′ and 14.3′ are also rotated by 180°—, one can imagine the output terminals 20.1 and 20.3 folded onto the output terminal 22.2 in this connection, just as the output terminals 22.1 and 22.3 are folded onto the output terminal 20.2. The phase rotation caused by the closed switches 28′, 30′, 32′ and 34′ can be compensated by adjusting the phase position at the RF generator unit 12.2′ accordingly. The HF generator unit 12.2′ is therefore operated out of phase relative to the generator units 12.1′ and 12.3′ when the switches 28′, 30′, 32′ and 34′ are closed.

In order to use the increased output current, an electrosurgical instrument can be connected on one side to one of the output terminals 20.1′, 22.2′ or 20.3′ and on the other side to one of the output terminals 22.1′, 20.2′ or 22.3′.

If—as shown in FIG. 6—only switch 28′ is closed and the other switches 30′, 32′ and 34′ are open, only generator modules 24.1′ and 24.2′ are connected in series on the output side, while the third generator module 24.3′ remains independent. In this case, an electrosurgical instrument requiring an increased output voltage can be connected between ports 20.1′ and 22.2′, while another electrosurgical instrument can be connected to output ports 20.3′ and 22.3′ to operate independently of the electrosurgical instrument connected to output ports 20.1′ and 22.2′.

For example, in order to be able to operate an electrosurgical instrument with an increased current requirement in combination with a second electrosurgical instrument, the generator modules 24.1′ and 24.2′ can also be connected in parallel on the output side, while the third generator module 24.3′ remains independent; see FIG. 7. In order to achieve this, the switches 28′ and 32′ are closed under the control of the control unit 26′, while the switches 30′ and 34′ remain open. In this way, the secondary windings 18.1′ and 18.2′ of the output transformers 14.1′ or 14.2′ are connected in parallel with each other. Here, too, the HF generator units 12.1′ and 12.2′ must be driven with a phase shifted by 180° with respect to each other—i.e., in phase opposition—so that the output transformers 14.1′ and 14.2′ are connected in parallel in phase on the output side by the switches 28′ and 32′. For example, the HF generator unit 12.2′ is operated with its phase position shifted by 180°.

An electrosurgical instrument with an increased current requirement can optionally be connected to the output connection 20.1′ or 22.2′ on the one hand and to the output connection 20.2′ or 22.1′ on the other hand. The second electrosurgical instrument can then be connected to output ports 20.3′ and 22.3′.

It is also possible to operate two of the three generator modules 24.1′, 24.2′ or 24.3′ in parallel—for example, the generator modules 24.1′ and 24.2′, as shown in FIG. 7—and to operate the third generator module in each case—in the example, the generator module 24.3′—in series with it. A corresponding example is shown in FIG. 8.

In principle, it is possible to operate any two of the three generator modules 24.1′, 24.2′ or 24.3′ in parallel or in series and to operate the third generator module independently—analogously to the examples shown in FIGS. 6 and 7. This can enable further variants in particular if the three generator modules 24.1′, 24.2′ or 24.3′ are not identical, but e.g. supply different output voltages.

### LIST OF REFERENCE NUMBERS

electrosurgical generator
12.1, 12.2, 12.3 HF generator units
14.1, 14.2, 14.3 output transformer
16.1, 16.2, 16.3 primary winding
18.1, 18.2, 18.3 secondary winding
20.1, 22.1, 20.2, 22.2, 20.3, 22.3 output terminals
24.1, 24.2, 24.3 HF generator module
26 control unit
28, 30, 32, 34 switch

1. An electrosurgical generator, comprising at least two HF generator units and at least two output transformers, each having a primary winding and a secondary winding, of which one of the primary windings is in each case connected to one of the HF generator units and one of the secondary windings is in each case connected to two output terminals for connecting an electrosurgical instrument, wherein one of the two output terminals is in each case provided for connecting a working electrode and the respective other one of the two output terminals is provided for connecting a neutral electrode, wherein, together with the associated output transformer and the corresponding output terminals, one HF generator unit respectively forms an HF generator module, wherein the HF generator modules are connected to a control unit, that is configured to at least provide the following operating modes:
a parallel mode in which the at least two output transformers are connected in parallel so that, via the output terminals, an electrosurgical instrument can be operated with a higher output current than the current that would be able to be provided by one HF generator module alone,
a series mode in which the at least two output transformers are connected in series so that, via the output terminals, an electrosurgical instrument can be operated with a higher output voltage than the voltage that would be able to be provided by one HF generator module alone, and
a multi instrument mode in which the output transformers are each connected independently of each other to output terminals for respectively one electrosurgical instrument.

2. An electrosurgical generator according to claim 1, wherein the electrosurgical generator with its control unit is adapted to additionally provide a multi-electrode mode, in which an output terminal of a generator module is electrically connected to an output terminal another generator module, which results in a total of three output terminals to each of which an electrode of an electrosurgical instrument is connected.

3. An electrosurgical generator according to claim 1, wherein at least three switches, that are connected to the output terminals in such a way that, for the parallel mode, through the closing of two of the three switches, two output terminals of different generator modules for a working electrode and two output terminals of different generator modules for a neutral electrode can respectively be electrically connected to each other, and further characterized in that, for the series mode, through the closing of the third switch, an output terminal for a working electrode of a generator module and an output terminal for a neutral electrode of another generator module can be electrically connected to each other, wherein, for the multi-instrument mode, all three switches are open.

4. An electrosurgical generator according to claim 3, wherein the control unit is operatively connected to at least three switches in order to switch them in accordance with the selected operating mode.

5. An electrosurgical generator according to claim 1, wherein the control unit for controlling the HF generator units is operatively connected to each of the at least two HF generator units.

6. An electrosurgical generator according to claim 1, wherein the control unit is configured to provide two versions of the multi-instrument mode, namely a first version in which the output terminals for connecting a neutral electrode are not electrically connected to each other, and a second version in which the output terminals for connecting a neutral electrode are electrically connected to each other.

7. An electrosurgical generator according to claim 1, wherein at least two output terminals for connecting a neutral electrode are connected to each other.

8. An electrosurgical generator according to claim 1, wherein the output transformers are in each case on their primary side connected to one of the HF generator units in such a way that, together with the associated output transformer, the respective HF generator unit forms a respective resonant circuit, wherein a primary winding of the respective output transformer is part of the respective resonant circuit.

9. An electrosurgical generator according to claim 1, wherein, in the “multi-electrode mode”, the at least two output terminals for connecting a neutral electrode are electrically connected to each other, and in that an electrode of an electrosurgical instrument is connected to the output terminals for connecting a neutral electrode.

10. An electrosurgical generator according to claim 1, wherein the control unit is configured to control the phase position between the currents and/or voltages output by the at least two HF generator modules 24.1 and 24.2.

11. An electrosurgical generator according to claim 1, wherein the HF generator modules are configured such that the maximum output voltage per generator module is 2,500 Vₚₑₐₖ.

12. An electrosurgical generator according to claim 1, wherein the HF generator modules are configured such that the maximum output current is 4.5 Aᵣₘₛ.

13. An electrosurgical generator according to claim 1, wherein the HF generator modules are configured such that their frequency range comprises 40 kHz to 1 MHz.
